(19) United States
(12) Patent Application Publication
(10) Pub. No.: US 2015/0010902 A1
(43) Pub. Date: Jan. 8, 2015

(54) Apparatus and Method for Monitoring Airborne Microorganisms in the Atmosphere

(71) Applicants: Kei Takenaka, Tokyo (JP); Hideyuki Noda, Tokyo (JP); Naoshi Itabashi, Tokyo (JP); Yoshiaki Yazawa, Tokyo (JP)

(72) Inventors: Kei Takenaka, Tokyo (JP); Hideyuki Noda, Tokyo (JP); Naoshi Itabashi, Tokyo (JP); Yoshiaki Yazawa, Tokyo (JP)

(73) Assignee: Hitachi, Ltd., Chiyoda-ku, Tokyo (JP)

(21) Appl. No.: 14/377,365

(22) PCT Filed: Feb. 8, 2012
(86) PCT No.: PCT/JP2012/052857

§ 371 Date: Sep. 25, 2014

(51) Int. Cl. G01N 33/569; G01N 1/22; G01N 21/64

(52) U.S. Cl. CPC G01N 33/569; G01N 21/6428; G01N 1/2214; G01N 2021/6439; G01N 2201/061; G01N 2201/0638; G01N 2001/245
USPC 435/5; 435/288.4; 435/30; 435/287.2; 435/7.1

(57) ABSTRACT

Disclosed is an apparatus for monitoring airborne microorganisms composed of: a chassis, which has a fan for flowing the air therein from the outside at a portion thereof and of which inside is spatially segmented by partition plates for performing a plurality of steps; a perforated plate, which is disposed at a portion of the chassis and has a plurality of nozzles for focusing split air flows passing through a plurality of spaces in a given direction; a capturing plate, which has a plurality of trapping surfaces at the positions opposite to the plurality of nozzles of the perforated plate; a capturing plate control part, which moves the capturing plate relative to the perforated plate; and an optical detection part for fluorescence generated from the microorganisms on the trapping surface of the capturing plate. The apparatus for monitoring airborne microorganism is characterized in that air containing microorganisms flows into some of the plurality of spaces segmented in the chassis, each of the plurality of trapping surfaces no the capturing plate has a pillar, and the capturing plate control part controls the position of the capturing plate; thereby, the air flows hit sequentially against the plurality of trapping surfaces of the capturing plate through the plurality of nozzles of the perforated plate from the plurality of spaces segmented in the chassis and the optical detection part detects sequentially fluorescence from the trapping surfaces of the capturing plate to detect the microorganisms for monitoring the presence or absence thereof.

### TECHNICAL FIELD

The present invention relates to an apparatus for monitoring airborne microorganisms and a method for continuously detecting airborne microorganisms to constantly monitor the presence of the airborne microorganisms.

### BACKGROUND ART

The infection spread of infectious diseases such as influenza and foot-and-mouth disease has come to a big social issue. It is considered that bacteria and viruses as pathogens are discharged into the atmosphere from affected human being and livestock, which in turn, may transmit to human being and livestock when being inhaled into their bodies. For this reason, attention is currently focused on a detection apparatus (apparatus for monitoring airborne microorganisms) for detecting airborne microorganism, such as bacteria and viruses, as a potent means to prevent the infection spread of these infectious diseases. A very small number of airborne microorganisms are found in the atmosphere, making it difficult to directly detect them. To solve this problem, conventional techniques for detecting these microorganisms involve generally two steps; (1) one of capturing the airborne microorganisms in the atmosphere (hereinafter, simply referred to as a capturing step) and (2) the other of detecting the captured microorganisms (hereinafter, simply referred to as a detecting step).

The capturing step usually uses a impaction technique, by which air containing particles injected from a nozzle, and the injected air is caused to hit against a trapping surface to adhere thereon and the detecting step generally uses a culture method, by which a mass of bacteria formed by culturing bacteria on culture media is visually measured; whereas another technique, an ATP method, which involves a step of detecting ATP (adenosine triphosphate) contained in bacteria to rapidly detect the captured bacteria, has been reported.

For instance, a portable-type airborne bacterium sampler disclosed in a patent literature 1 described below is an apparatus for capturing airborne bacteria on culture media by the impaction technique, which involves the steps of taking the culture media out of an apparatus after the completion of the capturing step and of measuring the bacteria by the culture method. In addition, a bacterium capturing carrier cartridge, carrier processing apparatus, and bacterium measurement method disclosed in a patent literature 2 described below are the method and apparatus for detecting he bacteria by the ATP method through the steps of: capturing the airborne bacteria on thermoplastic carrier, such as gelatin, by the impaction technique; and filtering the thermoplastic carrier liquefied with hot water to collect the bacteria on a filter.

### CITATION LIST

### Patent Literature

Patent literature 1: Japanese Unexamined Patent Application Publication No. 2000-304663
Patent literature 2: International Publication No. WO2009/157510

### SUMMARY OF INVENTION

### Technical Problem

To prevent infectious diseases such as influenza and foot-and-mouth disease from spreading, it is useful to capture airborne microorganisms such as bacteria and viruses as pathogens directly from the atmosphere for detection. The time required for a period from the capturing step to the detecting step, however, may increase the risk of creating new sources of infection because the affected human being and livestock, as well as potentially affected them, move to different places. To solve this problem, it is required for the airborne microorganism detection apparatus for preventing the spread of infectious diseases to shorten the time between the capturing and detections steps as possible. Moreover, it cannot be predicted whether the affected human being and livestock appear; thereby, it is required for the apparatus to have a function for constantly monitoring the presence or absence of airborne microorganisms.

However, for the aforementioned methods and apparatuses known according to conventional techniques, the step for detecting the microorganisms, such as bacteria and viruses, requires too long time (for instance, several days for the apparatus disclosed in the aforementioned patent literature 1, and several dozens of minutes for the apparatus disclosed in the aforementioned patent literature 2), making it difficult to meet these needs. Moreover, the aforementioned methods and apparatuses disclosed in the patent literatures 1 and 2 have such as problem that the culture media and thermoplastic carrier serving as trapping surfaces are disposable, making it difficult to have a constantly-monitoring function.

The object of the present invention, which has been attained in the light of the aforementioned problem with the conventional techniques, is to provide an apparatus for monitoring airborne microorganisms with a constantly-monitoring function and method, which involve the steps of capturing and detecting the airborne microorganisms at an airborne microorganism detection apparatus in a short time and continuously using the impaction technique.

### Solution to Problem

To attain the aforementioned object, the present invention provides the apparatus for monitoring airborne microorganisms composed of: a chassis, which has a fan for flowing the air therein from the outside at a portion thereof and of which inside is spatially segmented by partition plates for performing a plurality of steps; a perforated plate, which is disposed at a portion of the chassis and has a plurality of nozzles for focusing split air flows passing through a plurality of spaces in a given direction; a capturing plate, which has a plurality of trapping surfaces at the positions opposite to the plurality of nozzles of the perforated plate; a capturing plate control part, which moves the capturing plate relative to the perforated plate; and an optical detection part for fluorescence generated from the microorganisms on the trapping surface of the capturing plate. The apparatus for monitoring airborne microorganisms is characterized in that air containing microorganisms flows into some of the plurality of spaces segmented in the chassis, each of the plurality of trapping surfaces no the capturing plate has a pillar, and the capturing plate control part controls the position of the capturing plate; thereby, the air flows hit sequentially against the plurality of trapping surfaces of the capturing plate through the plurality of nozzles of the perforated plate from the plurality of spaces segmented in the chassis and the optical detection part detects sequentially fluorescence from the trapping surfaces of the capturing plate to detect the microorganisms for monitoring the presence or absence thereof.

Moreover, in the apparatus for monitoring microorganisms of the present invention, it is preferable that a substance, which binds specifically to the airborne microorganisms, has been bound to the pillars disposed at the plurality of trapping surfaces of the capturing. Furthermore, it is preferable that some of the plurality of spaces segmented in the chassis, excluding those, into which the air containing the microorganisms flows, have a spray part for spraying atomized liquid containing a fluorescent label into the internal air; alternatively, the spay part includes at least one of a fluorescent label spray part, a washing spray part for spraying atomized liquid containing the fluorescent label, which bonds to specific microorganisms, and a dissociation liquid spray part for spraying atomized low-pH liquid. The capturing plate is preferably a disk-shaped or rectangular plate, or a roll sheet; moreover the aforementioned perforated plate is preferably made of a disk-shaped metal sheet with 0.01 mm to 2 mm in thickness and 5 mm to 200 mm in diameter, and a plurality of through holes with circular cross section and 50 μm to 200 μm in hole diameter for forming the plurality of nozzles are radially from the center point of the disk. Furthermore, it is preferable that the areas of the pillars is one to 10 times those of the areas of the nozzles; the heights of the pillars are two times or more the intervals between the pillars and the nozzles; and the capturing plate is made of glass, quartz, resins (including polypropylene, polyethylene terephthalate, polycarbonate, polystyrene, acrylonitrile butadiene styrene resin, poly(methyl methacrylate) ester, and polydimethylsiloxane), or metals (including pure metals such as iron, aluminum, copper, tin, gold, and silver, and alloys of these metals). Furthermore, it is preferable that the diameters of the particles of liquid atomized for spraying from the spray part are 0.3 μm to 10 μm and the number densities thereof are 10⁶ to 10¹²/m³; and the positions of partition plates of the chassis is variable in the chassis; thereby, the ratio between the numbers of the plurality of nozzles for spraying the air containing microorganisms and of the plurality of nozzles for spraying the air containing atomized liquid may be changed.

Furthermore, to attain the aforementioned object, the method for monitoring airborne microorganisms of the present invention, by which the airborne microorganisms are captured for detecting thereof, is characterized in that it involves the steps of: spraying air on a plurality of trapping surfaces formed on the capturing plate for adhere the airborne microorganisms thereon; performing a given processing on the airborne microorganisms adhered on the trapping surfaces of the capturing plate; and detecting the airborne microorganisms adhered on the trapping surface, on which the given processing has been performed; a substance, which bonds specifically to the microorganisms, is adhered to the airborne microorganisms on the trapping surfaces; and the aforementioned steps are sequentially executed.

Additionally, in the aforementioned method for monitoring microorganisms of the present invention, it is preferable that the step of detecting the microorganisms detects optically fluorescence generated from the microorganisms and the step of performing the aforementioned processing spays atomized liquid containing a fluorescent label specifically binding to the certain kinds of microorganisms. Moreover, the method for monitoring the microorganism preferably further involves a step of spraying washing, which is atomized pure water or buffer, or a step of spraying dissociation liquid, which is atomized low-pH liquid, and as with the aforementioned step, the step is simultaneously and sequentially executed for monitoring the airborne microorganisms. Furthermore, the washing spray step or the dissociation liquid spray step is executed after the step of adhering airborne microorganisms.

### Advantageous Effects of Invention

The apparatus for monitoring airborne microorganisms and method of the present invention have very beneficial effects that the airborne microorganisms are captured and detected rapidly for constant monitoring the presence or absence thereof.

### BRIEF DESCRIPTION OF DRAWINGS

FIG. 1 is a perspective view including a partial perspective drawing illustrating the whole outline configuration of an apparatus for monitoring airborne microorganisms according to a first embodiment of the present invention present invention.

FIG. 2 is an enlarged view illustrating the configuration of a capturing part as a member of the apparatus for monitoring airborne microorganisms according to the first embodiment of the present invention.

FIG. 3 is a view explaining the detail of steps (capturing and labelling steps) in the apparatus for monitoring airborne microorganisms according to the first embodiment of the present invention.

FIG. 4 is a view explaining the detail of steps (washing and detecting steps) in the apparatus for monitoring airborne microorganisms according to the first embodiment of the present invention.

FIG. 5 is a view explaining the detail of a step (disassociation step) in the apparatus for monitoring airborne microorganisms according to the first embodiment of the present invention.

FIG. 6 is a view explaining the detail of a process of all the steps in the apparatus for monitoring airborne microorganisms according to the first embodiment of the present invention.

FIG. 7 is a perspective view including a partial perspective drawing illustrating the outline configuration of the apparatus for monitoring airborne microorganisms according to a second embodiment of the present invention.

FIG. 8 is a perspective view including a partial perspective drawing illustrating the outline configuration of the apparatus for monitoring airborne microorganisms according to a third embodiment.

### DESCRIPTION OF EMBODIMENTS

Changing the subject, as described above, the spread of infection with viruses such as avian influenza and foot-and-mouth viruses (hereinafter, simply referred to as viruses) has recently come to a big social issue and there is the need for preventing quickly the infection spread. To solve this problem, it has become imperative to prevent the infection spread by capturing and detecting airborne microorganisms. However, for the aforementioned reasons, it is required for apparatuses for capturing and detecting airborne microorganisms to prevent the infection spread to provide a function for executing the capturing step to the detecting step in short time as possible and a function for constantly monitoring the microorganisms.

To this end, the inventors of the present invention had discussed the structure for detecting the captured viruses in a short time and constantly monitoring the viruses, and finally achieved the following embodiments.

Hereinafter, by reference to the accompanying drawings, the embodiments of the present invention are explained in detail. The embodiments explained in the following paragraphs are illustrative only; accordingly, it goes without saying that any other aspects may be accepted by combining the following embodiments, or combining with or substituting by a publicly known or known technique.

In the descriptions, the terms, the apparatus for monitoring airborne microorganisms and method means mean the method and apparatus for detecting viruses, bacteria, yeast, protozoon, fungi, sporules, and pollen for monitoring the presence or absence thereof. It should be noted that for easy notation, all of generally defined microorganisms (bacteria, yeast, and fungi), as well as viruses, sporules, and pollen are referred together to as “microorganisms” in the description.

### First Embodiment

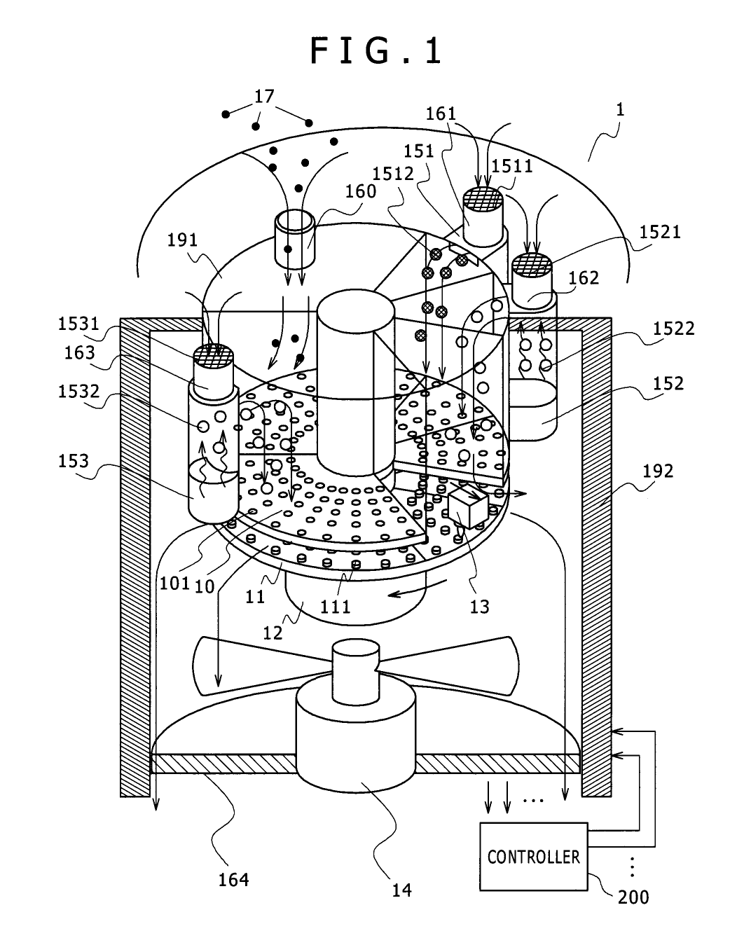

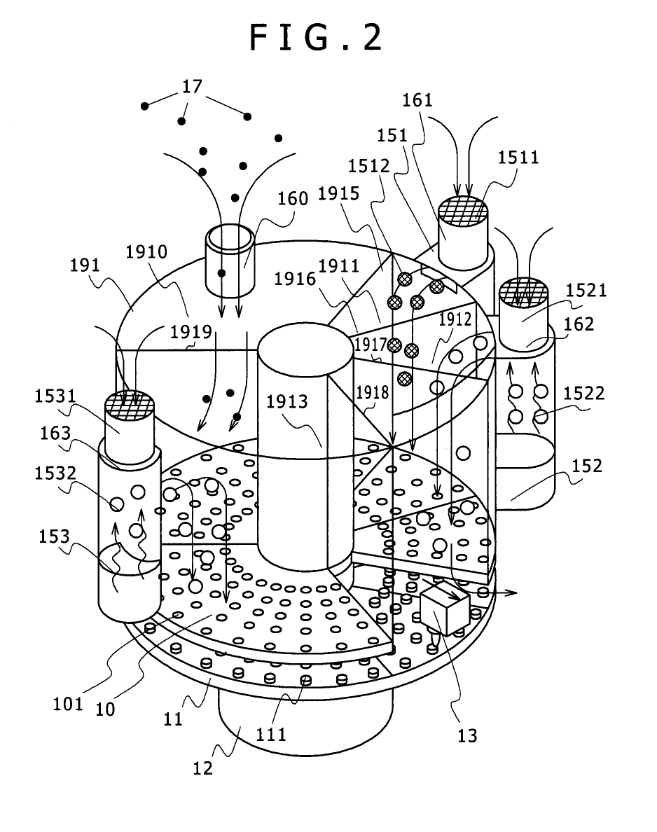

FIG. 1 is a view illustrating the outline configuration of an apparatus for monitoring airborne microorganisms according to a first embodiment of the present invention and FIG. 2 is an enlarged view showing a portion (including elements configured around the internal chassis 192) of the airborne microorganism monitoring apparatus.

First, the apparatus for monitoring airborne microorganisms 1 is composed of a cylindrical external chassis 192 and a cylindrical internal chassis 191 disposed on the top of the cylindrical external chassis; at the bottom of the internal chassis, a perforated plate 10 having a radial arrangement of a plurality of nozzles 101, a disk-shaped capturing plate 11 disposed on the lower side of the perforate plate 10 and having a plurality of pillars 111 for capturing the microorganisms passing through the aforementioned nozzles 101 on its upper surface, a capturing plate control part 12 for supporting the capturing plate 11 and controlling the movement thereof, and an optical detection part 13 for detecting optically the airborne microorganisms 17 captured on the he pillars 111 of the capturing plate 11. Moreover, on the lower side of the external chassis 192, a fan 14 and an outlet filter 164 have been disposed for taking air (containing microorganisms) in the chassis, and on the top of them, spray parts 151, 152, and 153 have been disposed for spraying atomized reagent and washing liquids. Openings, which serve as air sucking inlets 160, 161, 162, and 163 for taking the air flows in, have been formed on the top surfaces of the external chassis 192 and internal chassis 191. The sign 200 shown in the figure is the control part for controlling the movements of the aforementioned configuring members of the apparatus following the process of the steps timely, for which for instance a microcomputer including storage such as memory may be used.

The nozzles 101 formed on the perforated plate 10 and the pillars formed on the capturing plate 11 have been disposed at the same level of positions when viewed from above, each being disposed radially from the center point and in the direction of disk diameter with the same intervals, and equiangularly in the rotating direction. There is a relationship of concentric circle between these perforated plate 10 and capturing plate 11, and the capturing plate control part 12 controls the movement of the capturing plate 11 in the rotating direction to ensure that the nozzles 101 and the pillars 111 are opposite to each other.

The perforated plate 10 has a fan-like shape with the center angle equal θ to the superior angle (180°<θ<360°) and has been joined so as to form the bottom of the internal chassis 191. The space formed between the internal chassis 191 and the perforated plate 10 are segmented by internal partition plates 1915, 1916, 191, 1918, and 1919, a plurality of spaces 1910, 1911, 1912, and 1913. The individual spaces 1910 to 1913 serve as a microorganism space 1910, a fluorescent label space 1911, a washing space 1912, and a dissociation liquid space 1013 depending on the types of the microorganisms or mist passing through them; thereby, the partition plates 1915 to 1919 also serve as a microorganism space-fluorescent label space partition plate 1915, a fluorescent label space-washing space partition plate 1916, a washing space-detection part partition plate 1917, a detection part-dissociation liquid space partition plate 1918, and a dissociation liquid space-microorganism space partition plate 1919 depending on the segmented spaces.

On the top surfaces of the pillars 111 of the capturing plate 11, a substance (an antibody or the like), which binds specifically to the airborne microorganism 17, is bounded (or modifies). For this reason, when the airborne microorganisms 17 hit against on the top surfaces of the pillars 111, the airborne microorganisms 17 bind to the top surfaces of the pillars 111.

On the other hand, the spray parts 151 to 153 have a function of atomizing a supplied reagent. However, each of these spray parts are classified depending on the type of the sprayed reagent as follows. Specifically, the fluorescent label spray part 151 atomizes a liquid containing fluorescent label, which binds specifically to the airborne microorganisms 17 (fluorescent label mist 1512). The washing spray part 152 atomizes a washing for washing the fluorescent label adsorbed non-specifically on the pillars 111 (washing mist 1522). The disassociation liquid spray part 153 atomizes a disassociation liquid, which has effect of peeling the airborne microorganisms off from the pillars 111 (disassociation liquid mist 1532). The air sucking inlets 160 to 163 also serve as a microorganism sucking inlet 160, a fluorescent label sucking inlet 161, a washing sucking inlet 162, and a disassociation liquid sucking inlet 163 depending on the types of the microorganisms or mist to be mixed with the sucked air. The fluorescence label sucking inlet 161, the washing liquid sucking inlet 162, and the disassociation liquid sucking inlet have a filter 1511, 1521, and 1531 for removing any dust in the air, respectively. The capturing plate control part 12 is connected to the external chassis 192 by means of a beam-like structure (not indicated in FIGS. 1 and 2).

Although the detail will be described later, the step of detecting the airborne microorganisms 17 by the apparatus for monitoring airborne microorganisms 1 is executed as described below. First, an air flow occurs when a fan 14 rotates, and the air flow runs into the microorganism sucking inlet 160, the fluorescent label sucking inlet 161, the washing sucking inlet 162, and the disassociation liquid sucking inlet 163, accordingly. The flowing air and the airborne microorganisms 17 contained in the air hit against the top surfaces of the pillars 111 of the capturing plate 11 and is forced to flow in the direction of the sides of the pillars lllafter passing through the nozzles 101 of the perforated plate 10 via the microorganism sucking inlet 160 and the microorganism space 1910. At that time, if an inertia force of the airborne microorganisms 17 is too stronger than the force of the air flow, the airborne microorganisms 17 do not follow the air flow ad hit against the top surfaces of the pillars 111, causing them to be captured by the substance (which binds specifically to the airborne microorganisms 17) bound on the pillars 111.

On the other hand, the airborne microorganisms 17 contained in the air lowing via the fluorescent label sucking inlet 161 are removed when passing through the filter 1513. The filtered air passes together the fluorescent label mist 1512 generated from the fluorescent label spray part 151 through the nozzles 101 of the perforated plate 10 via the fluorescent label space 1911, hits against the top surfaces of the pillars of the capturing plate 11, and is forced to flow in the direction of the sides of the pillars 111. At that time, if the inertia force of the fluorescent label mist 1512 is too stronger than the force of flow, the fluorescent label mist 1512 does not follow the air flow and hit against the top surfaces of the pillars 111. Then, after hitting, the fluorescent label contained in the fluorescent label mist 1512 is specifically bound to the airborne microorganisms 17 captured on the top surfaces of the pillars 111. Similarly, the washing mist 1522 and the disassociation liquid mist 1532 are also captured on the top surfaces of the pillars 111. Thus, on the top surfaces of the individual pillars, the airborne microorganisms 17, the fluorescent label mist 1512, the washing mist 1522, or the disassociation liquid mist 1532 is supplied depending on the positions of these pillars.

On the other hand, when the capturing plate 11 is equiangularly (in the pitches in the direction in which the pillars rotate) stepwise-rotated by means of the aforementioned capturing plate control part 12, on the top surfaces of the pillars 111, sequentially executed may be the steps of: (1) capturing airborne microorganisms 17; (2) labelling the fluorescence on the airborne microorganisms 17 by supplying fluorescent label mist 1512; (3) washing out the fluorescent label adhered non-specifically to the airborne microorganisms 17 by supplying the washing mist 1522; (4) detecting the airborne microorganisms 17 on the pillars 111 by the optical detection part 13; and (5) disassociating the airborne microorganisms 17 y supplying the disassociation liquid mist 1532. Moreover, when the capturing plate 11 goes round, the process resumes the first step of capturing the airborne microorganisms 17. Thus, the plurality of the aforementioned steps are repeated so as to enable the test again.

Next, the aforementioned components are described in detail. The perforated plate 10 is made of a metal sheet with 0.01 mm to 2 mm in thickness and 5 mm to 200 mm in diameter. The diameters of the holes of the nozzles 101 formed on the perforated plate 10 depend on the diameters of the captured particles. Assuming that 90% or more of particulates with 300 μm in captured particle diameter are be to captured, the outer diameters need to be less than 200 μm and considering that the workability of the nozzles and the turbulent conditions of the air passing through the nozzles 101, the hole diameters are preferably, for instance 50 to 100 μm. The nozzles may be formed by any of the techniques such as etching, laser processing, electro-discharge machining, electron beam processing, and machining.

The capturing plate 11 is a disk-shaped plate having a plurality of pillars. The optimal value for the interval between the nozzles 101 and the top surfaces of the pillars 111, which varies with the diameters of the nozzles, is preferably ⅓ to 15 times the nozzle diameters, more preferably ½ to 5 times (25 to 500 μm). This means that if the diameters of the pillars 111 are too small, the airborne microorganisms 17 are difficult to hit against the top surfaces of the pillars 111. In contrast, when they are too large, the larger detection range is needed, making the time required for detection longer. Lower the height of the pillars 111, easier the processing of them is; however, part of the air flow from the nozzles 101 flows into the top surfaces of the adjacent pillars 111, making it difficult for the airborne microorganisms 17 to hit against the top surfaces of the pillars 111. The result of the detailed discussion by the inventors made it clear that to ensure that the airborne microorganisms passing through the nozzles 101 hit against the top surfaces of the pillars 111 and the airborne microorganisms 17 on the top surfaces of the pillars 111 are effectively detected, the diameters of the pillars are preferably one to 10 times the diameters of the nozzles 101 and the height of the pillars 111 is preferably two times or more the interval between the nozzles 101 and the top surfaces of the pillars 111.

The capturing plate 11 is made preferably of silicone, glass, quartz, resins (including polypropylene, polyethylene terephthalate, polycarbonate, polystyrene, acrylonitrile butadiene styrene resin, or acryls such as poly(methyl methacrylate) ester, and polydimethylsiloxane). The pillars 111 may be formed, depending on the materials, by any of techniques such as for instance etching for silicone, glass, and quartz, or hot-embossing, mold injection, and transfer printing for resins.

As mentioned above, the capturing plate control part 12 provides a function of rotating the capturing plate 11 stepwise; however, in this embodiment, a sensor is disposed for detecting the position of the capturing plate 11 to reduce the displacement between the pillars 111 of the capturing plate 11 and the nozzles 101 of the perforated plate 10. After rotating the capturing plate 11 stepwise, adjustment between the positions of the pillars of the capturing plate 11 and the nozzles 101 of the perforated plate 10 is made based on information provided by the sensor. The optical detection part 13 for detecting the airborne microorganisms 17 on the top surfaces of the pillars 111 may be used for the sensor.

The optical detection part 13 is composed of : an optical system for detecting fluorescence having a light source of exciting light for exciting the fluorescent label; an optical detector for detecting the fluorescence generated from the fluorescent label; a lens system for focusing the exciting light from the light source and fluorescence form the fluorescent label; an optical filter for selecting the wavelengths of the exciting light and fluorescence; and spatial filters (pinholes) for removing stray light, an alignment control mechanism for aligning the focus of the optical system for detecting fluorescence on the top surfaces of the pillars 111, and a radial-direction movement control mechanism for moving the optical detection part 13 in the radial direction of the capturing plate 11. These movement control mechanisms enable the airborne microorganisms 17 to be detected by detecting the fluorescence generated form the fluorescent label binding to the airborne microorganisms 17 captured on the top surfaces of the pillars 111 while rotating e optical detection part 13 in the radial direction of the capturing plate 11.

Each of the fluorescent label spray part 151, the washing spray part 152, and the disassociation liquid spray part 153 provides a function of nebulizing for atomizing reagents as mentioned above. The reagent atomized into mist by these spray parts is sucked and mixed with the air, and supplied on the top surfaces of the pillars 111 of the capturing plate 11 using the air flow.

It is preferable that the diameters of the particles of liquid atomized for spraying from the spray part are 0.3 μm to 10 μm and the number densities thereof are 10⁶ to 10¹²/m^{3.}; and the positions of partition plates of the chassis is variable in the chassis.

For the filters 1511, 1521, 1531 and the outlet filter 164, HEPA filters (High Efficiency Particulate Air Filters) are used. These filters 1511, 1521, and 1531 suppress the airborne microorganisms 17 and other particles from mixing into the sucked air, preventing abnormal test result. The outlet filter 164 is used for removing any virus aggregate and the mist of a reagent, which have not been captured on the capturing plate 11.

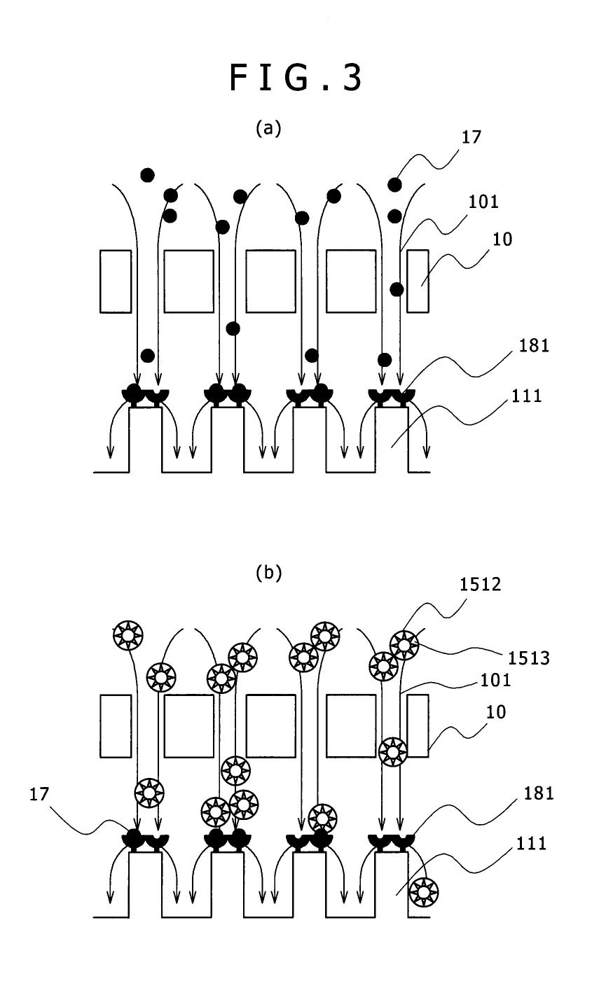

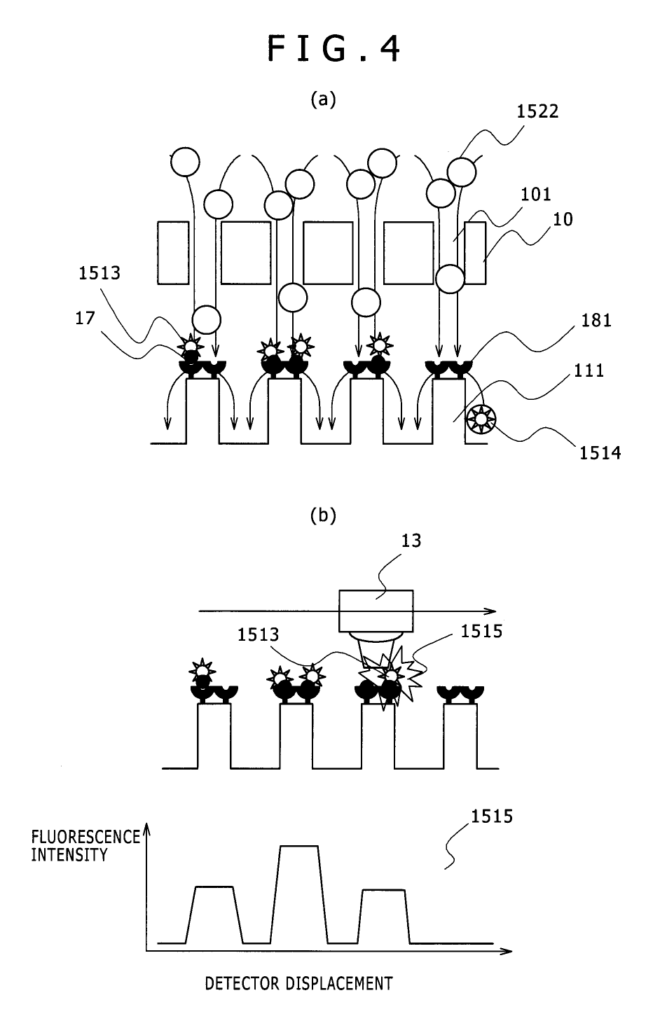

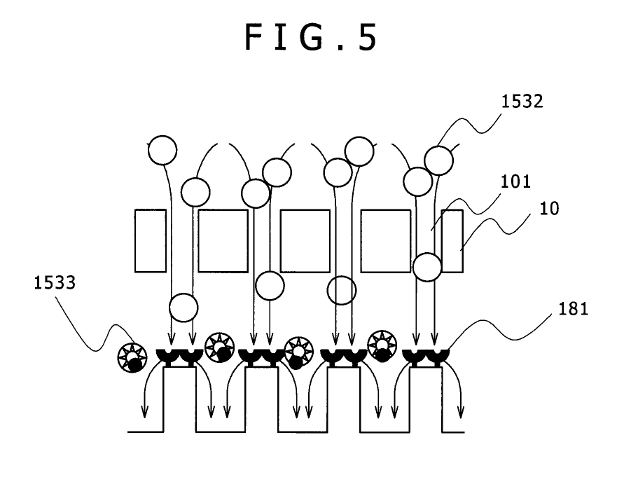

Next, by reference to FIGS. 3(a) to 3(b), FIG. 4(a) to (b), and FIG. 5, the step for testing is explained in detail.

Capturing step (See FIG. 3):

Together with sir sucked by a fan 14 (FIG. 1), the airborne microorganisms 17 contained the air pass through the nozzles 101 of the perforated plate 10. The sucked air, after hitting against the top surfaces of the pillars 111 of the capturing plate 11, is forced to flow on the sides of the pillars 111, however, as mentioned above, the airborne microorganisms 17 hit against the top surfaces of the pillars 111 by means of the inertia force thereof. Since the top surfaces of the pillars 111 is modified with an antibody 181, which binds specifically to an antigen existing on the surfaces of the airborne microorganism 17, the airborne microorganisms 17, which have hit against the top surfaces of the pillars 111 bind specifically to the top surfaces of the pillars 111 through antibody response. The technique by which the antibody 181 is bound to the top surfaces of the pillars 111 is commonly known and includes for instance the binding technique using non-specific adsorption, the binding technique using silane coupling treatment, and the binding technique using a certain type of linker.

Labelling step (See FIG. 3(b)):

The fluorescent label mist 1512 generated from the fluorescence label spray part 151 (FIG. 1) is mixed with the air sucked by the fan 14 (FIG. 1) and passes through the nozzles 101 of the perforated plate 10. At that time, the sucked air, after hitting against the top surfaces of the pillars 111 of the capturing plate 11, is forced to flow on the sides of the pillars 111, however the fluorescent label mist 1512 hits against the top surfaces of the pillars 111 through the inertia force thereof. Since the fluorescent label mist 1512 contains the fluorescent label 1513 (an antibody labeled with a fluorescent dye) therein, which binds specifically to the airborne microorganisms 17, the airborne microorganisms 17 binding the antibody 181 on the top surfaces of the pillars 111 and the fluorescent label 1513 are bound specifically to each other in the aforementioned capturing step.

As mentioned above, antigen-antibody reaction using minute fluorescent label mist 151 (φ0.3 μm to 10 μm) has the following two advantages.
(Advantage 1): diffusion time shortened by minimizing the diffusion distance: to cause the airborne microorganisms 17 and the fluorescent label 1513 to be bound to each other, these two substances need to approach to each other to the sufficient distance. Theoretically, the time required for substance to travel a certain distance is proportional to the square of the distance. Comparing the antibody-antigen reaction in a standard reaction container (assuming that the radius of the liquid is 1 mm, and the depth of the liquid is 1 mm) and the antibody-antigen reaction in minute mist, for instance, the distance between the airborne microorganisms 17 and the fluorescent label 1513 is about max. 1 mm apart in a microtiter plate, while it is about max. 10 μm (equivalent to the diameter of the mist) in the minute mist. For this reason, the time required for hitting is 1/10000 times that for reaction in the microtiter plate.
(Advantage 2): shortened reaction time by further concentering the density of the viruses: For the reaction, in which a substance A and a substance B are bound to each other to form AB, the time required for binding is proportional to the concentrations of these two substances. Similarly to the above description, comparing the antibody-antigen reaction in a standard reaction container (assuming that one airborne microorganism contained in the liquid with 1 mm in radius and 1 mm in depth) and the antibody-antigen reaction in minute mist (assuming that one airborne microorganism in the liquid with 1 mm in depth), for instance, the concentration of the airborne microorganisms 17 in the minute mist is about 1×10⁶ times as higher as that of the airborne microorganisms 17 in the standard reaction container. For this reason, the reaction time is reduced by 1/10⁶.
   Washing step (See FIG. 4(a)):

The washing mist 1522 generated by the washing spray part 152 (FIG. 1) s mixed with the air sucked by the fan 14 (FIG. 1) and passes through the nozzles 101 of the perforated plate 10. The sucked air, after hitting against the top surfaces of the pillars 111 of the capturing plate 11, is forced to flow on the sides of the pillars 111; however, the washing mist 1522 hits against the top surfaces of the pillars 111 through the inertia force thereof. The washing mist 1522 hitting against the top surfaces takes the fluorescent label 1514 adsorbed on the top surfaces of the pillars 111. And then, when the washing mist 1522 repeatedly hit against the top surfaces, part of it overflows from the top surfaces of the pillars 111; thereby, the fluorescent label 1514 taken in may be removed from the top surfaces of the pillars 111. On the other hand, since the airborne microorganisms 17 binding specifically to the antibody on the top surfaces of the pillars 111 is bound strongly to the fluorescent label 1513 binding specifically to e airborne microorganisms, they remain on the top surfaces of the pillars 111. Low-concentration of buffer or pure water is suitable for a washing.

Detecting step (See FIG. 4 (b)):

While moving the optical detection part 13 in the radial direction of the capturing plate 11 (FIG. 1), the intensity of the airborne microorganism is measured on the top surfaces of the pillars 111. Specifically, if the airborne microorganisms 17, to which the fluorescent label 1513 is bound, are found on the top surfaces of the pillars 111, the optical detection part 13 measures the strong fluorescence 1515 while the pillars 111 move. For this reason, Based on the measured intensity of fluorescence, the number of the captured airborne microorganisms 17 may be counted.

Disassociating step (See FIG. 5):

The disassociation liquid mist 1532 generated from the disassociation liquid spray part 153 (FIG. 1) is mixed with the air sucked by the fan 14 (FIG. 1) and passes through the nozzles 101 of the perforated plate 10. The sucked air, after hitting against the top surfaces of the pillars 111, is forced to floe on the sides of the pillars 111; however the disassociation liquid mist 1532 hit against the top surfaces of the pillars 111 of the capturing plate 11 through the inertia force thereof and the disassociation liquid mist 1532 hitting against the top surfaces takes in the airborne microorganisms 17, which are bound to the antibody 181 on the top surfaces of the pillars 111. The disassociation liquid disassociates a substance binding to the airborne microorganisms 17 through antigen-antibody reaction with low-pH liquid. When the disassociation liquid mist 1532 repeatedly hits against the tope surfaces, part of it overflows from the top surfaces of the pillars 111; thereby, the airborne microorganisms 17 may be removed from the top surfaces of the pillars 111.

When the airborne microorganisms 17 binding to the antibody 181 on the pillars 111 are removed, as mentioned above, the top surfaces of the pillars 111 return to their original states and resuming the capturing step makes it possible to repeatedly capturing and detection of the airborne microorganisms 17. The operations at the individual parts in the aforementioned steps are executed by a program stored in memory, etc., in the aforementioned microcomputer.

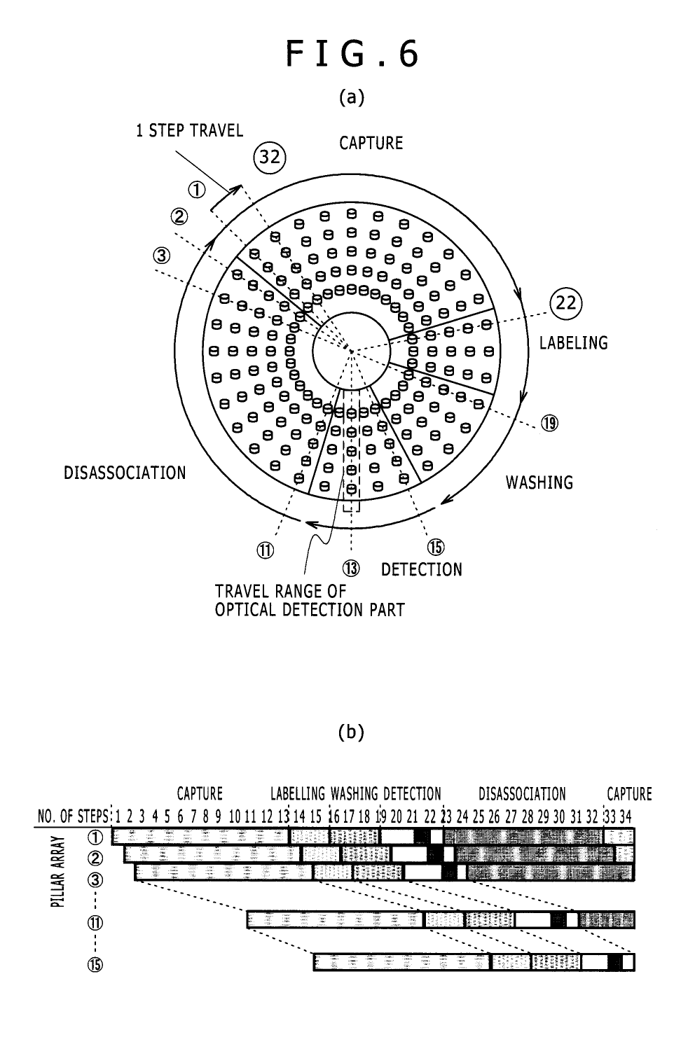

Next, by reference to FIGS. 6(a) and 6(b), the mechanism for the method for constantly monitoring airborne microorganisms at the apparatus 1 for monitoring airborne microorganisms. FIG. 6(a) shows the position of the capturing plate 11 at any point. The circled numbers in the figures show an array of pillars existing on a radius of the capturing plate 11. In the following paragraphs, no circled numbers used in the figures are used and instead, they are referred to as the array of pillars 1, 2 . . . . In the apparatus 1 for monitoring airborne microorganisms of the present invention, the partition plates 1915 to 1919 (FIG. 2) segment a plurality of steps to be executed; therefore, the arrays of pillars 1, and 23 to 32 are included in the capturing step, the arrays of pillars 20 to 22 are included in the labelling step, the arrays of pillars 16 to 19 are included in the washing step, the arrays of pillars 12 to 15 are included in the detecting step, and the arrays of pillars 2 to 11 are included in the disassociating step for executing their corresponding steps at this time point. Among them, in the detecting step, only on the array of pillars 13, fluorescence is measured at the optical detection part 13. Upon the measurement of fluorescence, to avoid contact of the optical detection part 13 with other parts of the apparatus 1 for monitoring airborne microorganisms while the optical detection part 13 moves in the radius direction of the capturing plate 11, given spaces are left at the positions before and after the array of pillars 13.

After a certain time has elapsed from this time point, the capturing plate 11 rotationally moves by means of the function of the capturing plate control part 12 by one step. Specifically, the array of pillars 2 moved to the position of the arrays of pillars 1 and the array of pillars 3 moves to the position of the array of pillars 2. Thus, as shown in FIG. 6(b), each of steps is executed while the individual arrays of pillars are slightly shifted, namely the steps necessary for detecting airborne microorganisms are simultaneously, in parallel, sequentially, and continuously, enabling constant monitoring.

Moreover, the time required for executing each step depends on the types of the target microorganisms and used reagents; however, since the time required for executing each of steps is determined by setting the positions of the partition plates 1915 to 1919 in the apparatus 1 for monitoring airborne microorganisms of the present invention, varying the position of the partition plate allows the time required for executing each of steps to be adjusted if necessary.

### Second Embodiment

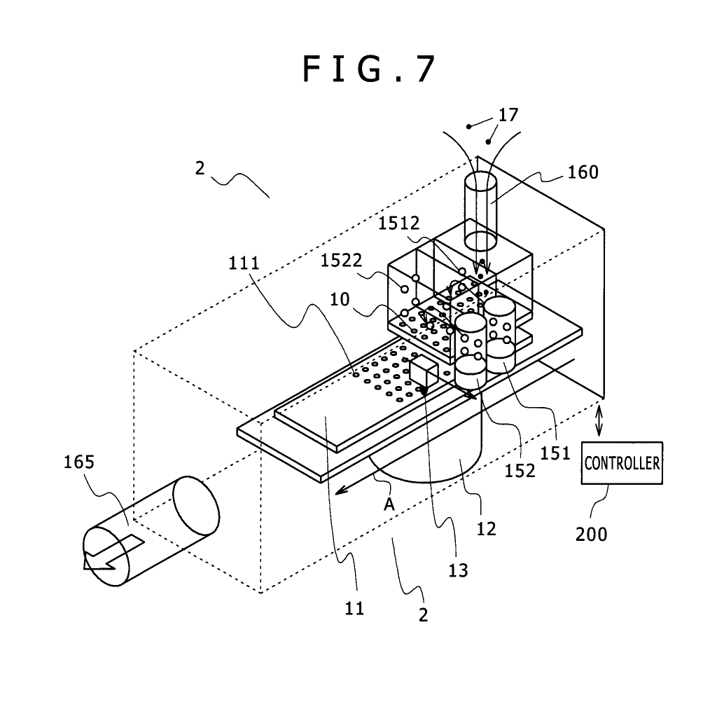

FIG. 7 shows the outline configuration of an apparatus for monitoring airborne microorganisms according to another aspect (second embodiment) of the present invention and the second embodiment illustrated in this figure is mainly intended to test the airborne microorganisms in an easier way. Specifically, the large differences of the apparatus for monitoring airborne microorganisms according to the second embodiment of the present invention from that according to the first embodiment of the present invention are the shape of the capturing plate 11 and the method for controlling the movement of the capturing plate in the capturing plate control part 12. In the figure, the same signs are indicated on the corresponding components of the apparatus according to first embodiment and their detailed explanations of them are omitted.

More specifically, the capturing plate 11 is made of a rectangular plate having a plurality of pillars 111, and the capturing plate control part 12 moves linearly stepwise in the direction indicated by an arrow head A in the figure. Norte that the apparatus a for monitoring airborne microorganisms is composed of a capturing part, a labeling part, and a washing part as with the apparatus 1 for monitoring airborne microorganisms according to the aforementioned first embodiment of the present invention.

Focusing on the movements of the apparatus for monitoring airborne microorganisms 17, the airborne microorganisms pass together the air sucked by a fan (not indicated) through the nozzles of the perforated plate 10 via an air sucking inlet 160. The airborne microorganisms 17 passing through the nozzles hit against the top surfaces of the pillars 111 of the capturing plate 11 as with the apparatus according to the first embodiment of the present invention. The capturing plate 10 is moved stepwise by a distance of the pillars 111 in the direction indicated by the arrow head A by the capturing plate control part 12. On the top surfaces of the pillars 111, against which the airborne microorganisms 17 hit, the washing mist 1522 generated from the fluorescent label spray part 152 hits together with the fluorescent label mist 1512 generated from the fluorescence label spray part 151 as with the apparatus according to the first embodiment of the present invention. Moreover, by detecting the fluorescence of the fluorescent label 1513, which binds specifically to the airborne microorganisms 17 on the top surfaces of the pillars 111 at the optical detection part 13, the presence or absence of the airborne microorganisms 17 may be easily determined. However, according to the second embodiment, no disassociating step is not executed; thereby, to continue the test, the capturing plate 11 must be replaced a new one after every detection step ends.

### Third Embodiment

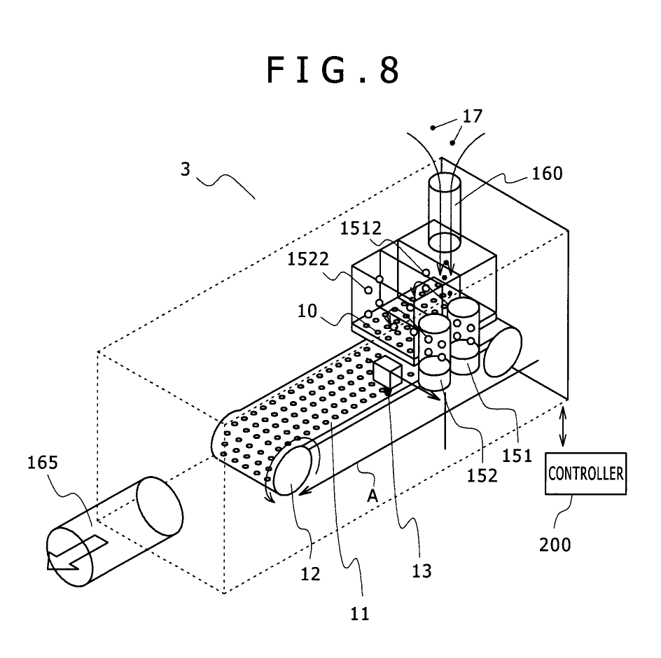

FIG. 8 shows the outline configuration of an apparatus for capturing airborne microorganisms according to further another aspect (third embodiment) of the present invention. According to the apparatus for monitoring airborne microorganisms according to the third embodiment of the present invention, the presence or absence of airborne microorganisms may be monitored for a longer time period than that of the apparatus 2 for monitoring airborne microorganisms 17 according to the aforementioned second embodiment of the present invention. The differences of the apparatus according to the third embodiment of the present invention from that according to the second embodiment are the capturing plate 11, which has been shaped in roll sheet and the capturing plate control part 12 rotates as if it rolls up the roll-sheet capturing plate 11; thereby, the capturing plate 11 moves in the direction indicated by the arrow head A shown in the figure. Namely, since the capturing plate 11 has been shaped into roll sheet, it is possible to repeatedly capture and detect the airborne microorganisms 17 as with the apparatus according to the aforementioned first embodiment; thereby, the capturing plate 11 may be used for a longer period than that of the capturing plate 11 according to the second embodiment of the present invention and has a disassociation liquid spray part (See 153 in FIG. 1) (not indicated in the figure). It should be noted that the same signs are used for the corresponding components to those of the apparatuses according to the aforementioned embodiments in the figure and the detail explanation of them are omitted.

It should be noted that according to the aforementioned first, second, and third embodiments, the fluorescence label is bound specifically to the airborne microorganisms to detect the airborne microorganisms. Thus, the use of the fluorescence label makes it easier to identify the airborne microorganisms and improves considerably the detection sensitivity; however, the liquid containing the fluorescent label need to be replenished depending on the remaining amount if necessary and in some cases, the detecting step must be temporarily stopped.

To this end, hereinafter, the method and configuration for detecting airborne microorganisms without using the fluorescence label are explained. Generally, the airborne microorganisms having cells, in which fluorescent substances such as NADH (nicotinamidea denine dinucleotide reduced), NADPH (Reduced nicotinamide adenine dinucleotide disodium salt), and flavoprotein are contained. Accordingly, by disposing a function (means) for irradiating an exciting light for exciting these fluorescent substances (ultraviolet ray for NADH and NADPH, and blue light for flavoprotein), as well as a function (means) for detecting fluorescence (blue for NADH and NADPH, and green for flavoprotein) generated from these fluorescent substances in the aforementioned optical detection part 13, the airborne microorganisms may be continuously detected with no need for replenishing the fluorescence label, namely with no need for temporal stop of the apparatus for maintenance mentioned above.

### LIST OF REFERENCE SIGNS

1, 2, 3 . . . microorganism detection apparatus, 10 . . . perforated plate, 11 . . . capturing plate, 12 . . . capturing plate control part, 13 . . . optical detection part, 14 . . . fan, 101 . . . nozzle, 111 . . . pillar, 151-153 . . . spray parts, 105 . . . filter, 106 . . . holder, 107 . . . inner periphery outlet, 108 . . . outer periphery outlet, 109 . . . virus aggregate, 112 . . . rough capturing substrate, and 123 . . . column.

1-15. (canceled)

16. An apparatus for monitoring airborne microorganisms comprising:
a chassis, which has a fan for flowing the air therein from the outside at a portion thereof and of which inside is spatially segmented by partition plates for performing a plurality of steps;
a perforated plate, which is disposed at a portion of the chassis and has a plurality of nozzles for focusing split air flows passing through a plurality of spaces in a given direction;
a capturing plate, which has a plurality of trapping surfaces at the positions opposite to the plurality of nozzles of the perforated plate;
a capturing plate control part, which moves the capturing plate stepwise relative to the perforated plate; and
an optical detection part for fluorescence generated from the microorganisms on the trapping surface of the capturing plate,
wherein air containing microorganisms flows into some of the plurality of spaces segmented in the chassis,
wherein the capturing plate control part controls the position of the capturing plate; thereby, the air flows hit sequentially against the plurality of trapping surfaces of the capturing plate through the plurality of nozzles of the perforated plate from the plurality of spaces segmented in the chassis, and
wherein the optical detection part detects sequentially fluorescence from the trapping surfaces of the capturing plate, on which the airflows containing the microorganisms from some of the plurality of spaces segmented in the chassis to detect the microorganisms for monitoring the presence or absence thereof.

17. The apparatus for monitoring the airborne microorganisms according to claim 16, wherein each of the plurality of trapping surfaces of the capturing plate has a pillar, and a substance, which binds specifically to the airborne microorganisms, has been bound to the pillars disposed at the plurality of trapping surfaces of the capturing plate.

18. The apparatus for monitoring the airborne microorganisms according to claim 17, wherein some of the plurality of spaces segmented in the chassis, excluding those, into which the air containing the microorganisms flows, have spray parts for spraying atomized liquid containing a fluorescent label into the internal air.

19. The apparatus for monitoring the airborne microorganisms according to claim 18, wherein the spray part includes at least one of a fluorescent label spray part for spraying atomized liquid containing fluorescent label, which binds specifically to the certain kinds of microorganism, a washing spray part for spraying atomized pure water or buffer, and a disassociation liquid spray part for spraying atomized low-pH liquid.

20. The apparatus for monitoring the microorganisms according to claim 19, wherein the capturing plate is a disk-shaped or rectangular plate, or a roll sheet.

21. The apparatus for monitoring the microorganisms according to claim 20, wherein the perforated plate is made of disk-shaped metal sheet with 0.01 to 2 mm in thickness and 5 to 200 mm in diameter, and a plurality of through holes with 50 to 200 μm in hole diameter and circular cross section for forming the nozzles are arranged radially from the center point of the disk.

22. The apparatus for monitoring the airborne microorganisms according to claim 21, wherein the areas of the pillars one to 10 times those of the nozzles, and the heights of the pillars are two times or more the interval between the pillars and the nozzles.

23. The apparatus for monitoring the airborne microorganisms according to claim 22, wherein the capturing plate is made of glass, quartz, resins (including polypropylene, polyethylene terephthalate, polycarbonate, polystyrene, acrylonitrile butadiene styrene resin, poly(methyl methacrylate) ester, and polydimethylsiloxane), or metals (including pure metals such as iron, aluminum, copper, tin, gold, and silver, and alloys of these metals).

24. The apparatus for monitoring the airborne microorganisms according to claim 23, wherein the diameters of the particles of liquid atomized for spraying from the spray part are 0.3 to 10 μm and the number densities thereof are 10⁶ to 10¹²/m³.

25. The apparatus for monitoring the airborne microorganisms according to claim 24, wherein the positions of partition plates of the chassis is variable in the chassis; thereby, the ratio between the numbers of the plurality of nozzles for spraying the air containing microorganisms and of the plurality of nozzles for spraying the air containing atomized liquid may be changed.

26. A method for monitoring airborne microorganisms by capturing the airborne microorganisms for detection, comprising the step of:
spraying air on a plurality of trapping surfaces formed on the capturing plate for adhering the airborne microorganisms thereon;
performing a given processing on the airborne microorganisms adhered on the trapping surfaces of the capturing plate; and
detecting the airborne microorganisms adhered on the trapping surface, on which the given processing has been performed,
wherein a substance, which bonds specifically to the microorganisms, is adhered to the airborne microorganisms on the trapping surfaces; and the aforementioned steps are simultaneously and sequentially executed.

27. The method for monitoring airborne microorganisms according to claim 26, wherein the step of detecting the microorganisms detects optically fluorescence generated from the microorganisms.

28. The method for monitoring airborne microorganisms according to claim 27, wherein the step for performing the given processing sprays atomized liquid containing fluorescence, which binds specifically to the certain kinds of microorganisms.

29. The method for monitoring airborne microorganisms according to claim 28, wherein a washing spray step for spraying atomized pure water or buffer or a disassociation liquid spray step for spraying atomized low-pH liquid further included, and the airborne microorganisms are monitored by executing a process of these steps as with that of the aforementioned steps simultaneously and sequentially.

30. The method for monitoring airborne microorganisms according to claim 29, wherein the washing spray step or the disassociation liquid spray step is executed after the step of adhering the airborne microorganisms.
